(19) United States
(12) Patent Application Publication
(10) Pub. No.: US 2017/0049710 A1
(43) Pub. Date: Feb. 23, 2017

(54) NOVEL FORMULATIONS AND METHODS

(71) Applicants: Leo RUBIN, Suffern, NY (US); Shaker MOUSA, Wynantskill, NY (US)

(72) Inventors: Leo RUBIN, Suffern, NY (US); Shaker MOUSA, Wynantskill, NY (US)

(73) Assignee: AVANT GARDE THERAPEUTICS AND TECHNOLOGIES, LLC, Suffern, NY (US)

(21) Appl. No.: 15/183,496

(22) Filed: Jun. 15, 2016

Related U.S. Application Data
(63) Continuation of application No. 13/704,526, filed on Nov. 13, 2013, now abandoned.
(60) Provisional application No. 61/355,283, filed on Jun. 16, 2010.

(51) Int. Cl. A61K 9/51; A61K 47/48; A61K 38/22

(52) U.S. Cl. CPC A61K 9/5161; A61K 38/2278; A61K 9/5153; A61K 47/4823

(57) ABSTRACT

Nanoparticles comprising VIP and their use in treating, e.g., pulmonary hypertension. Such nanoparticles provide improved delivery of VIP and allow for acute treatment and optionally for sustained release of VIP in a patient.

This application is a continuation of U.S. application Ser. No. 13/704,526, filed Dec. 14, 2012, which claims benefit to International Application PCT/US2011/040704 filed on Jun. 16, 2011, which claims priority from U.S. Provisional Application 61/355,283 filed Jun. 16, 2010, the contents of which are herein incorporated by reference in their entirety.

### TECHNICAL FIELD

The field relates to nanoparticles comprising vasoactive intestinal peptides (VIP), and to their use in treatment of pulmonary hypertension, and vascular and neurological disorders.

### BACKGROUND OF THE INVENTION

Pulmonary hypertension (PH or PHT) is an increase in blood pressure in the pulmonary artery, pulmonary vein, and/or pulmonary capillaries. It is a very serious condition, potentially leading to shortness of breath, dizziness, fainting, decreased exercise tolerance, heart failure, pulmonary edema, and death. It can be one of five different groups, classified by the World Health Organization as follows:

### WHO Group I—Pulmonary Arterial Hypertension (PAH)

a. Idiopathic (IPAH)
b. Familial (FPAH)
c. Associated with other diseases (APAH): collagen vascular disease (e.g. scleroderma), congenital shunts between the systemic and pulmonary circulation, portal hypertension, HIV infection, drugs, toxins, or other diseases or disorder.
d. Associated with venous or capillary disease
   Pulmonary arterial hypertension involves the vasoconstriction or tightening of blood vessels connected to and within the lungs. This makes it harder for the heart to pump blood through the lungs, much as it is harder to make water flow through a narrow pipe as opposed to a wide one. Over time, the affected blood vessels become both stiffer and thicker, in a process known as fibrosis. This further increases the blood pressure within the lungs and impairs their blood flow. In addition, the increased workload of the heart causes thickening and enlargement of the right ventricle, making the heart less able to pump blood through the lungs, causing right heart failure. As the blood flowing through the lungs decreases, the left side of the heart receives less blood. This blood may also carry less oxygen than normal. Therefore it becomes more and more difficult for the left side of the heart to pump to supply sufficient oxygen to the rest of the body, especially during physical activity.
   WHO Group II—Pulmonary Hypertension Associated with Left Heart Disease
a. Atrial or ventricular disease
b. Valvular disease (e.g. mitral stenosis)
   In pulmonary venous hypertension (WHO Group II) there may not be any obstruction to blood flow in the lungs. Instead, the left heart fails to pump blood efficiently out of the heart into the body, leading to pooling of blood in veins leading from the lungs to the left heart (congestive heart failure or CHF). This causes pulmonary edema and pleural effusions. The fluid build-up and damage to the lungs may also lead to hypoxia and consequent vasoconstriction of the pulmonary arteries, so that the pathology may come to resemble that of Group I or III.
   WHO Group III—Pulmonary Hypertension Associated with Lung Diseases and/or Hypoxemia
a. Chronic obstructive pulmonary disease (COPD), interstitial lung disease (ILD)
b. Sleep-disordered breathing, alveolar hypoventilation
c. Chronic exposure to high altitude
d. Developmental lung abnormalities
   In hypoxic pulmonary hypertension (WHO Group III), the low levels of oxygen may cause vasoconstriction or tightening of pulmonary arteries. This leads to a similar pathophysiology as pulmonary arterial hypertension.
   WHO Group IV—Pulmonary Hypertension Due to Chronic Thrombotic and/or Embolic Disease
a. Pulmonary embolism in the proximal or distal pulmonary arteries
b. Embolization of other matter, such as tumor cells or parasites
   In chronic thromboembolic pulmonary hypertension (WHO Group IV), the blood vessels are blocked or narrowed with blood clots. Again, this leads to a similar pathophysiology as pulmonary arterial hypertension.

### WHO Group V—Miscellaneous

Treatment of pulmonary hypertension has proven very difficult Antihypertensive drugs that work by dilating the peripheral arteries are frequently ineffective on the pulmonary vasculature. For example, calcium channel blockers are effective in only about 5% of patients with IPAH. Left ventricular function can often be improved by the use of diuretics, beta blockers, ACE inhibitors, etc., or by repair/replacement of the mitral valve or aortic valve. Where there is pulmonary arterial hypertension, treatment is more challenging, and may include lifestyle changes, digoxin, diuretics, oral anticoagulants, and oxygen therapy are conventional, but not highly effective. Newer drugs targeting the pulmonary arteries, include endothelin receptor antagonists (e.g., bosentan, sitaxentan, ambrisentan), phosphodiesterase type 5 inhibitors (e.g., sildenafil, tadalafil), prostacyclin derivatives (e.g., epoprostenol, treprostenil, iloprost, beroprost), and soluble guanylate cyclase (sGC) activators (e.g., cinaciguat and riociguat). Surgical approaches to PAH include atrial septostomy to create a communication between the right and left atria, thereby relieving pressure on the right side of the heart, but at the cost of lower oxygen levels in blood (hypoxia); lung transplantation; and pulmonary thromboendarterectomy (PTE) to remove large clots along with the lining of the pulmonary artery.

Heart failure and acute myocardial infarction are common and serious conditions frequently associated with thrombosis and/or plaque build-up in the coronary arteries.

Vasoactive intestinal peptide (VIP) is a peptide hormone containing 28 amino acid residues, produced in many areas of the human body including the gut, pancreas and suprachiasmatic nuclei of the hypothalamus in the brain. In humans, the vasoactive intestinal peptide is encoded by the VIP gene. Various synthetic forms of VIP or VIP from other mammalian sources are known. VIP causes vasodilatation, lowers arterial blood pressure, stimulates myocardial contractility, increases glycogenolysis and relaxes the smooth muscle of trachea, stomach and gall bladder. VIP is a potent dilator of the pulmonary and coronary arteries, and has great potential to reduce pulmonary arterial hypertension and at the same time enhance cardiac function. VIP is also known to dilate the cardiac arteries and to enhance cardiac function. VIP is therefore useful to treat acute myocardial infarction and to treat heart failure resulting from myocardial infarction. It thus has potential to help patients having conditions such as pulmonary hypertension, cardiac insufficiency, heart failure, and acute myocardial infarction. To date, however, it has not been used as a therapeutic because it has a half-life (T_{1/2}) in the blood of less than two minutes.

There is thus an unmet need for improved treatments for pulmonary hypertension, particularly pulmonary arterial hypertension, for cardiac insufficiency due to partial or complete blockage of coronary arteries and/or damage due to myocardial infarction, for example acute or congestive heart failure and acute myocardial infarction. There is moreover a need for a means of sustaining levels of VIP for longer periods of time, e.g. to treat such conditions.

### SUMMARY OF THE INVENTION

The invention provides VIP nanoparticles, wherein the VIP is encapsulated or immobilized by a bioabsorbable polymer, for example, having any of the following characteristics
a. Wherein the polymer comprises chitosan.
b. Wherein the polymer comprises poly(lactic-co-glycolic acid) (PLGA) or polylactic acid (PLA), e.g., PLGA having 50/50 co-polymerization of D,L-lactic acid and glycolic acid.
c. Wherein the polymer comprises chitosan crosslinked using glutaraldehyde.
d. Wherein the polymer comprises chitosan linked to bile acids.
e. Wherein the polymer comprises chitosan linked to PLGA, e.g., using glutaraldehyde as crosslinker.
f. Any of the foregoing wherein the nanoparticles have an average diameter of 50-1000 nm, e.g., 100-500 nm or 50-250 nm.
g. Any of the foregoing wherein the nanoparticles have a zeta potential of 10-100 mV, e.g. at least 40 mV, for example at least 60 mV, e.g. 50-80 mV.
h. Any of the foregoing wherein the nanoparticle comprises a second pharmacologically active ingredient
i. Any of the foregoing wherein the VIP is covalently linked to the bioabsorbable polymer.
j. Any of the foregoing wherein the VIP is encapsulated within the bioabsorbable polymer.
k. Any of the foregoing wherein the VIP is in a matrix created by the bioabsorbable polymer.

In one example, the VIP nanoparticles are made from VIP and the following components:

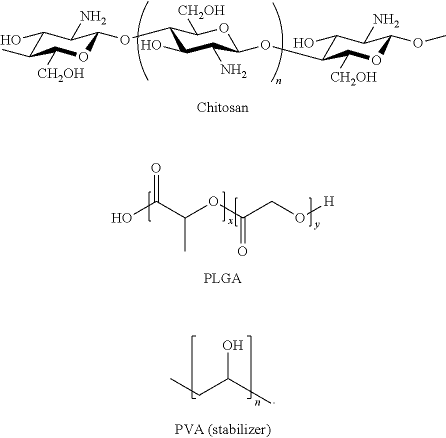

In one example, the nanoparticles have these components in approximately the following amounts:

| Components | Approx Amount | |
|---|---|---|
| of the | (% w/w) in the | |
| formulation | nanoformulation | Role in the formulation |
| Chitosan | 50-70%, e.g. 60% | Component of the nanocarrier |
| PLGA | 20-30%, e.g. 25% | Component of the nanocarrier |
| VIP | 10-20%, e.g. 15% | Active ingredient (chemically |
| | | conjugated to the nanoparticles) |

The contents of the nanoparticles are confirmed using, e.g. HPLC and LC/MS. The nanoparticle formulations may be sterilized using conventional means, e.g., filtration, gamma radiation. The nanoparticles are optionally coated with bile salt, lipid, PEG for improved delivery.

In one embodiment, the invention provides a method for treating pulmonary hypertension, e.g., pulmonary arterial hypertension, comprising administering an effective amount of a VIP-nanoparticle formulation to a patient in need thereof, wherein the VIP-nanoparticle comprises a bioabsorbable polymer, for example as described above.

In another embodiment, the invention provides a method for treating cardiac insufficiency, e.g., heart failure, angina, or acute myocardial infarction, comprising administering an effective amount of a VIP-nanoparticle formulation to a patient in need thereof, wherein the VIP-nanoparticle comprises a bioabsorbable polymer, for example as described above.

In a specific example of the foregoing methods, the VIP-nanoparticle administered comprises chitosan-PLGA nanoparticles encapsulating VIP.

In another example, the VIP-nanoparticle administered comprises chitosan nanoparticles encapsulating VIP with glutaraldehyde as a cross linker. Other cross-linkers may be used. In yet another example, the VIP-nanoparticle administered comprises chitosan-PLGA nanoparticles encapsulating VIP alone. Such examples of VIP nanoparticles may utilize a process that includes gelation/conjugation of preformed biodegradable polymers.

In yet another example, the VIP-nanoparticle administered includes chitosan-PLGA nanoparticles immobilizing VIP. Alternatively, the VIP-nanoparticles administered comprises chitosan-PLGA nanoparticles immobilizing VIP as well as chitosan-PLGA nanoparticles encapsulating VIP.

In another example, the VIP-nanoparticle comprises VIP covalently linked to chitosan or chitosan-PLGA nanoparticles.

In another example, the present invention also includes methods for treating pulmonary hypertension, comprising administering an effective amount of a VIP nanoparticle formulation to a patient in need thereof. It is contemplated by the present invention that an effective amount of a VIP nanoparticle formulation may be used to treat pulmonary arterial hypertension. It is further contemplated by the present invention that a VIP nanoparticle formulation may be administered in conjunction with: endothelin receptor antagonists (e.g., bosentan, sitaxentan, ambrisentan), phophodiesterase type 5 inhibitors (e.g., sildenafil, tadalafil), prostacylin derivatives (e.g., epoprostenol, treporostenil, iloprost, beroprost), and soluble guanylate cyclase (sGC) activators (e.g., cinaciguat and riociguat).

In yet another example, the present invention contemplates the use of a VIP nanoparticulate formulation to treat a patient in need thereof. It is contemplated by the present invention that a VIP nanoparticulate formulation may be used to treat cardiac insufficiency, e.g., heart failure, angina, or acute myocardial infarction. It is also contemplated by the present invention that a VIP nanoparticulate formulation may be used to treat pulmonary hypertension, e.g. pulmonary arterial hypertension.

### BRIEF DESCRIPTION OF THE FIGURES

FIG. 1 depicts a flow chart diagram for the synthesis of chitosan nanoparticles encapsulating VIP.

FIGS. 2A, 2B, 2C, and 2D show a size measurement for Chitosan nanoparticles encapsulating VIP.

FIGS. 3A and 3B and depict flow charts for the synthesis of chitosan PLGA-nanoparticles.

FIGS. 4A and 4B shows a size measurement of chitosan nanoparticles encapsulating VIP by DLS.

FIGS. 5A and 5B shows a size measurement of gluteraldehyde crosslink chitosan nanoparticles encapsulating VIP.

FIG. 6 depicts a Zeta potential measurement of chitosan nanoparticles encapsulating VIP.

FIG. 7 depicts Zeta potential measurement of void chitosan nanoparticles.

FIG. 8 shows Zeta potential measurement of gluteraldehyde crosslinked chitosan nanoparticles encapsulating VIP.

FIG. 9 shows Zeta potential measurement of gluteraldehyde crosslinked void chitosan nanoparticles.

FIG. 10 depicts the HPLC of VIP.

FIG. 11 depicts a Q1 scan of VIP, m/z from 450 to 1700.

FIG. 12 depicts a Q2 scan of product ion of VIP (M+5H+).

FIG. 13 depicts the results of VIP of the effects of topically applied VIP derivatives in CAM model on Branch Points Formation

### DETAILED DESCRIPTION

VIP, as used herein includes any peptide or peptide analogue having VIP activity, e.g., capable of binding VPAC₁ or VPAC₂, esp. VPAC₁, e.g. selected from
l. Human VIP, e.g His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn-NH 2;
m. VIP from other mammals, e.g., porcine VIP;
n. Active fragments or derivatives, of human or other mammalian VIP, e.g. comprising at least residues 11-27 of VIP.
o. Human or other mammalian VIP precusor protein;
p. Analogues of VIP, e.g. (Ala(2,8,9,11,19,22,24,25,27,28)VIP, (Ala(2,8,9,11,19,24-28))VIP, (Ala(2,8,9,16,19,24))VIP, (Ala(2,8,9,16,19,24,25))VIP, e.g., as disclosed in Igarashi H, et al. J Pharmacol Exp Ther. 2005 October; 315(1):370-81; Tyr(9),Dip(18)WIP(1-28), e.g., as disclosed in Tams, et al, Mol Pharmacol. 2000 November; 58(5):1035-41; R15, 20, 21, L17]-VIP-GRR as disclosed in Ohmori, et al. Life Sci. 2006 Jun. 6; 79(2):138-43; stearyl-norleucine-vasoactive intestinal peptide (stearyl-Nle17-VIP), as described in Gozes, et al, Endocrinology. 1994 May; 134(5):2121-5; Ro 24-9881 or Ro 25-1553, e.g., as described in O'Donnell et al, J Pharmacol Exp Ther. 1994 September; 270(3):1282-8; or analogues as disclosed in U.S. Pat. No. 7,094,755 and U.S. Pat. No. 4,835,252; other linear and cyclic VIP analoges as known in the art; the contents of the foregoing publication and patents being incorporated herein by reference;
q. Prodrugs, e.g., physiologically hydrolysable and acceptable esters of esters of VIP;
   in free or pharmaceutically acceptable salt form. Human VIP is preferred. Human VIP may be produced, e.g. recombinantly or synthetically, preferably recombinantly, and may be provided, e.g., in the form of the amide.

Administration routes include, but are not limited to intravenous, intra-arterial, intracardiac, subcutaneous, intramuscular, orally, intrapulmonary (e.g., by inhalation), intradermal, topically or rectally. The formulation may be for immediate release, e.g., via intravenous, intra-arterial, or intracardiac injection, or may be in the form of a sustained release depot formulation, e.g., a depot comprising a bioerodable polymer comprising the VIP nanoparticles of the invention, for example for subcutaneous or intramuscular injection, resulting in release of VIP over a period of days or weeks.

In one embodiment the VIP nanoparticles can be used in a drug-eluting metal or bioresorbable stent, e.g., for patients having had or at risk of acute myocardial infarction, e.g., for insertion in the coronary artery.

In a further embodiment, the VIP-eluting stents are also useful for patients with a history of stroke or transient ischemic attacks or patients otherwise at risk of stroke, e.g., for placement in the carotid artery, or for patients having pulmonary hypertension, e.g. for placement in the pulmonary artery.

In one embodiment, administration is by a pump activated by a signal, which releases the nanoparticles into the bloodstream. In one embodiment the signal is generated when pulmonary arterial pressure rises above a given level, e.g., greater than 30, for example, greater than 40 mmHg, as measured by an electronic pressure transducer linked to a cannula in the pulmonary artery. In another embodiment, the signal is generated when oxygen levels in the blood drop below a certain level, e.g., % SpO2 below 90, e.g., below 85 as measured by a pulse oximeter.

In one embodiment, the particles provide a sustained release which allows the VIP to affect gene expression.

The VIP nanoparticles of the invention may be administered in conjunction with, or adjunctive to, the normal standard of care for pulmonary hypertension or cardiac insufficiency or other cardiovascular or neurological disorders, for example in conjunction with one or more of:
r. Drugs selected from the group consisting of endothelin receptor antagonists (e.g., bosentan, sitaxentan, ambrisentan), phosphodiesterase type 5 inhibitors (e.g., sildenafil, tadalafil), prostacyclin derivatives (e.g., epoprostenol, treprostenil, iloprost, beroprost), and soluble guanylate cyclase (sGC) activators (e.g., cinaciguat and riociguat).
s. Diuretics, e.g., hydrochlorothiazide
t. Anticoagulants, e.g., Coumadin, aspirin
u. Calcium channel blockers, e.g., amlodipine
v. Beta-blockers, e.g. metoprolol
w. ACE inhibitors, e.g. captopril, enalapril
x. Nitrates, e.g. nitroglycerin
y. Inhaled beta-agonists, corticosteroids, and/or anticholinergics
z. Other antihypertensives

Various methods of synthesizing VIP-nanoparticles are provided. For example, a single emulsion process may produce chitosan-PLGA nanoparticles encapsulating VIP. In yet another example, a process involving gelation/conjugation of preformed biodegradable polymers produces 1) chitosan nanoparticles encapsulating VIP with and without glutaraldehyde as a cross-linker; or 2) chitosan-PLGA nanoparticles encapsulating VIP. Other cross-linkers may be used.

In yet another example, a process involving chemical bonding of VIP on the surface of chitosan-PLGA nanoparticles produces 1) chitosan-PLGA nanoparticles immobilizing VIP or 2) chitosan-PLGA nanoparticles immobilizing VIP and additionally including chitosan-PLGA nanoparticles encapsulating VIP.

For example, in one embodiment, PLGA and VIP are first immersed in a 1% PVA solution and chitosan. They are then stirred and sonicated. Then a dialysis step is performed. After a dialysis step occurs, PLGA-chitosan nanoparticles encapsulating VIP are produced. Then in the final step, the nanoparticles may then have a chitosan layer cross-linked with glutaraldehyde. Other cross-linkers may be used.

An entrapment efficiency may also be measured. The entrapment efficiency may be calculated to be the total amount of VIP in the nanoparticles/initial concentration of VIP added to make the formulation×100.

### EXAMPLES

### Example 1

### Synthesis of VIP Encapsulated Nanoparticles

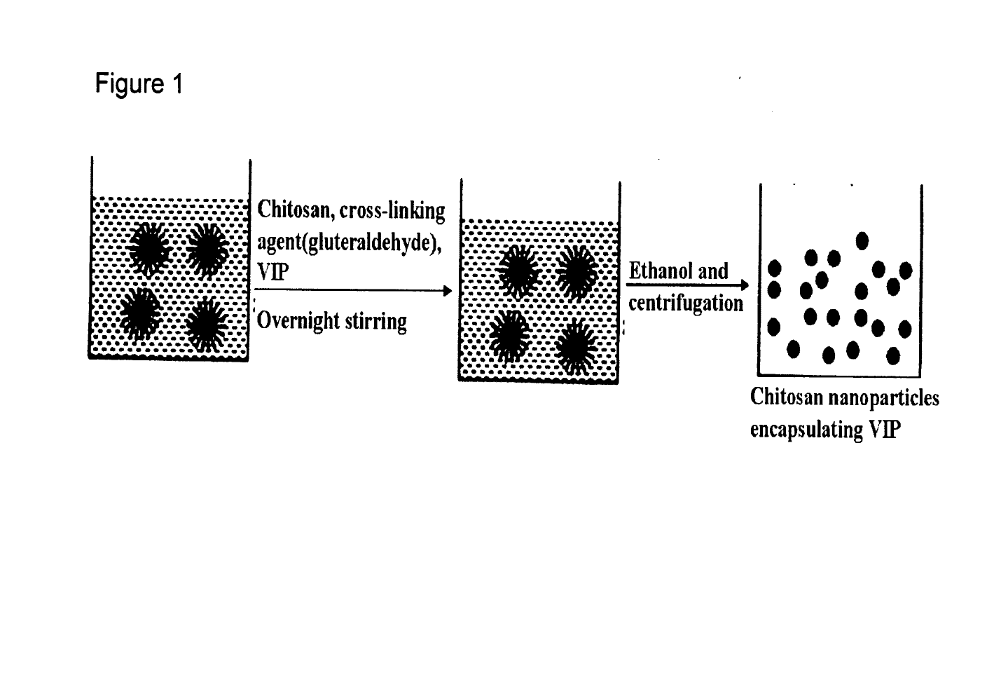

Chitosan nanoparticles encapsulating VIP are produced using a reverse micellar method as shown in FIG. 1. Chitosan polymer and VIP are added to 0.1M AOT/hexane (AOT-Aerosol OT is used as a surfactant) solution to form reverse micelles. Bifunctional reagent gluteraldehyde is added to this reverse micelles system as a cross-linking agent. The chemical cross-linking of chitosan polymers with gluteraldehyde occurs by Schiff's reaction of aldehyde groups on gluteraldehyde and amino groups on the chitosan chain. Finally nanoparticles are separated out by high speed centrifugation.

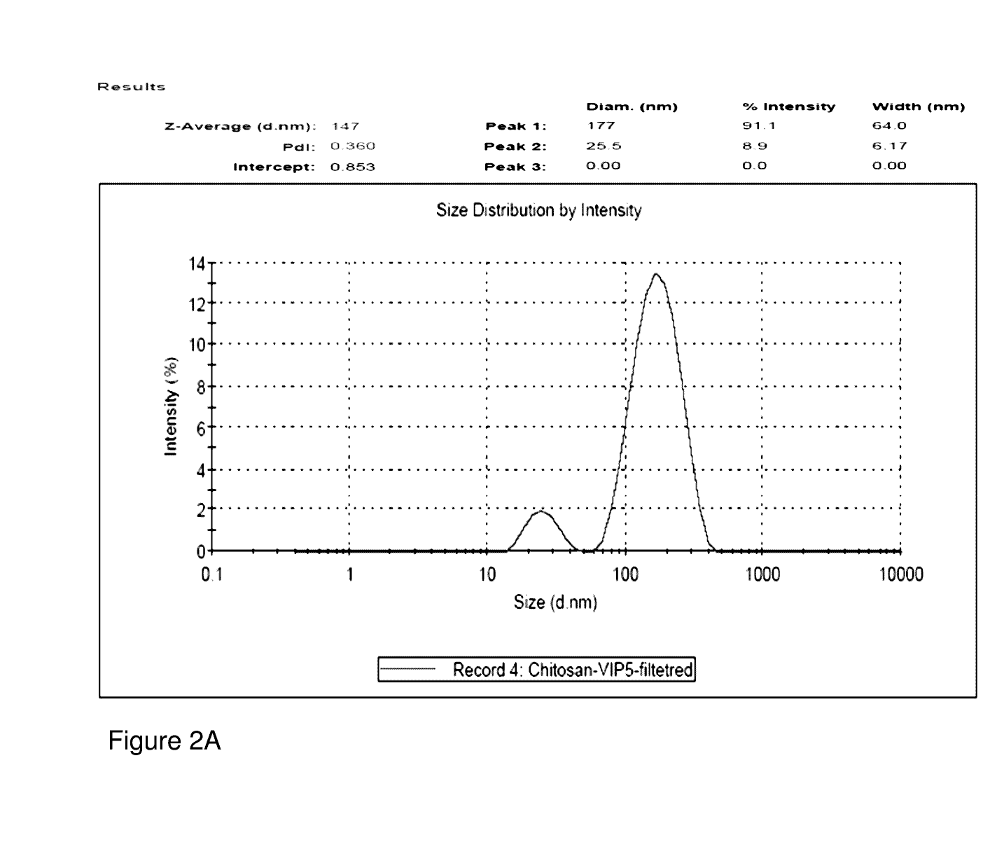

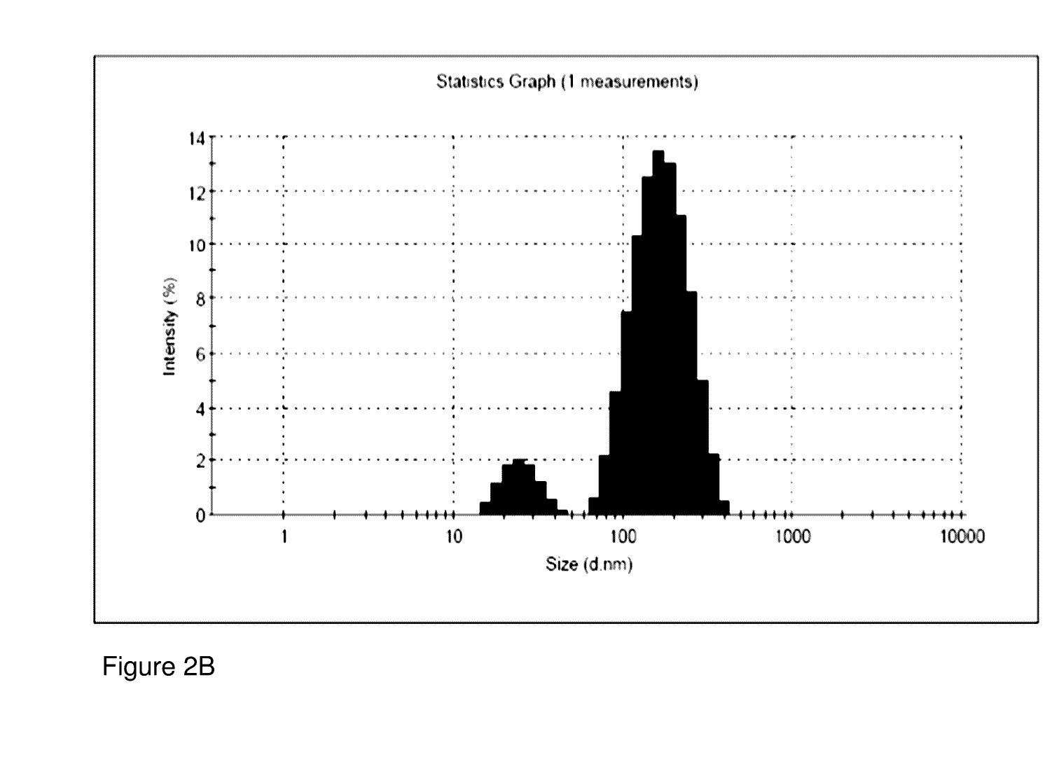

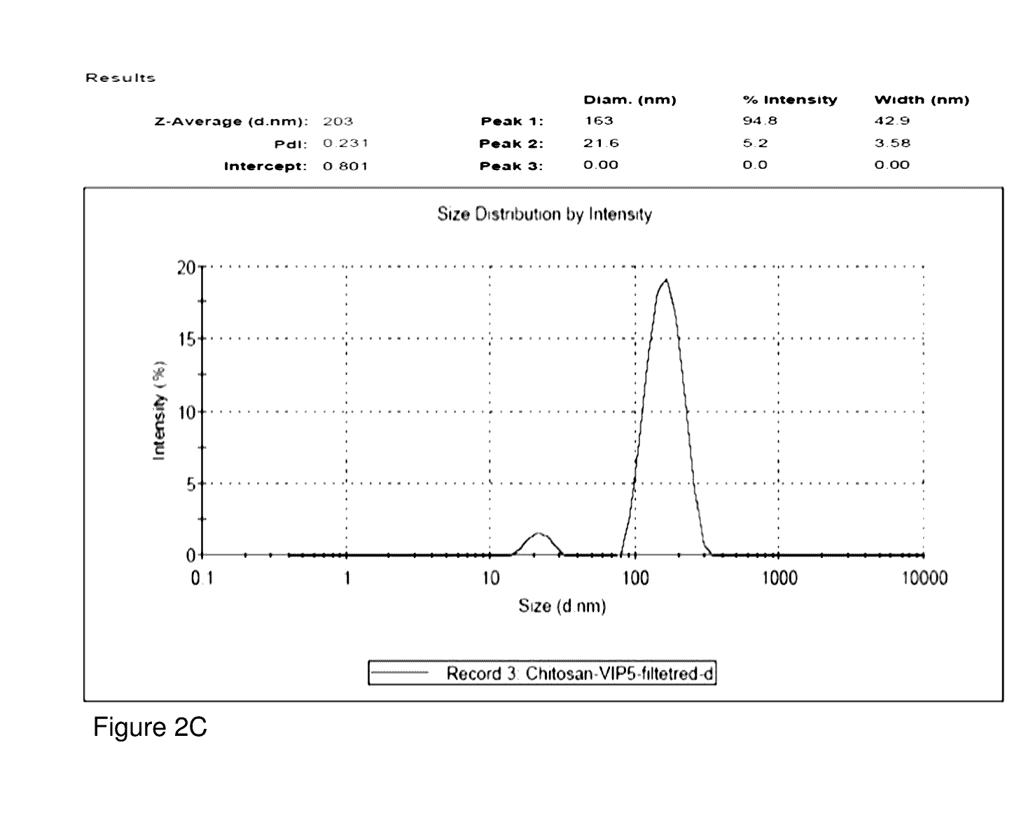

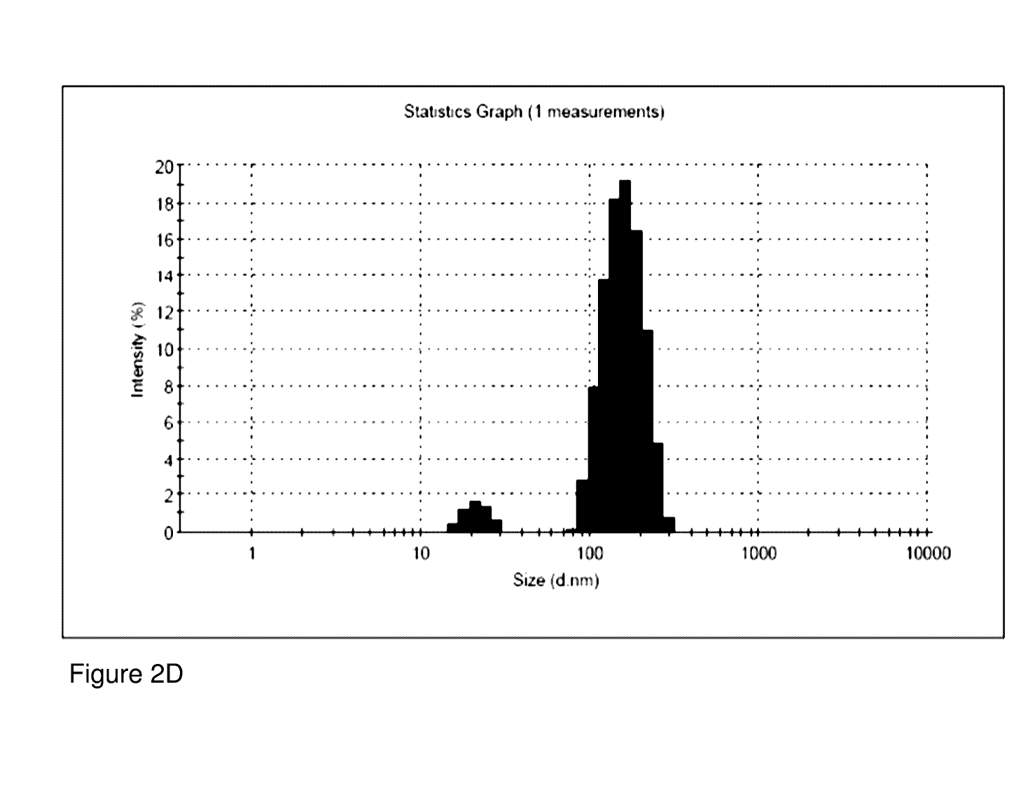

In FIGS. 2A, 2B, 2C, and 2D, these charts depict representative diagrams for size measurement for Chitosan nanoparticles encapsulating VIP. In these examples, nanoparticles are optimized as to size, and entrapment efficiency to get an optimum formulation with maximum loading.

### Example 2

### Synthesis of Chitosan-PLGA Nanoparticles

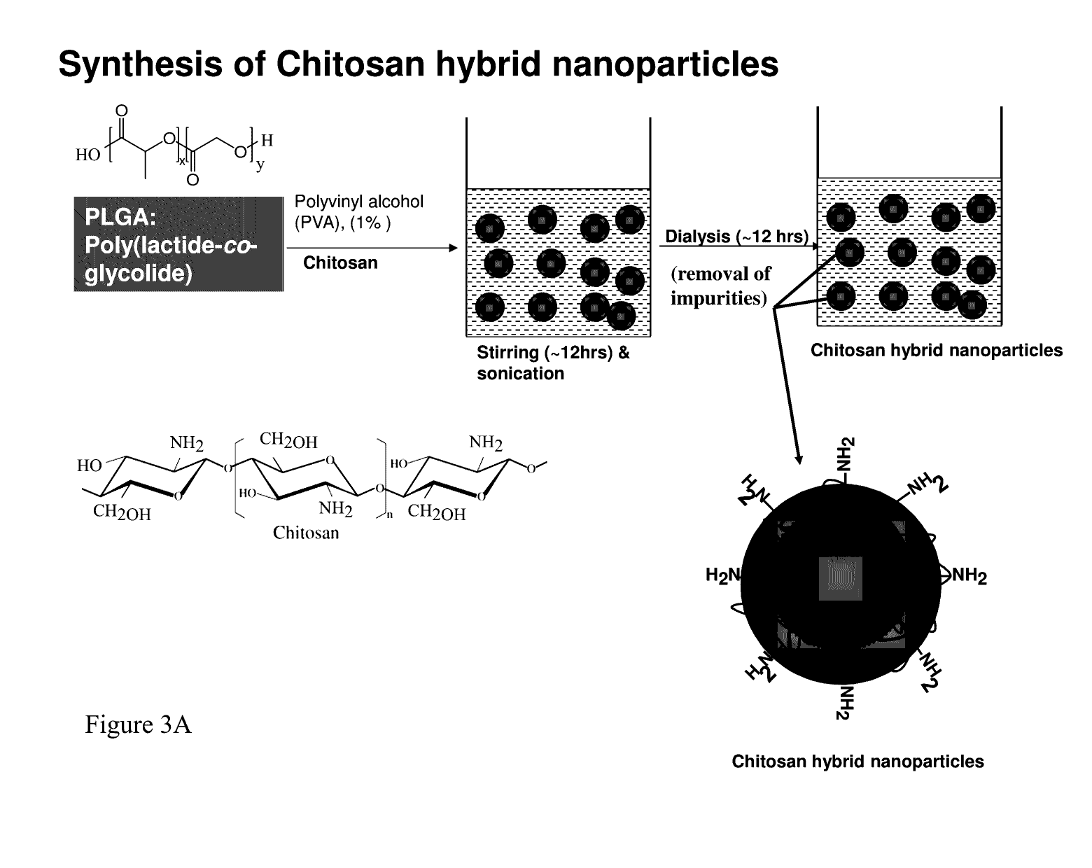

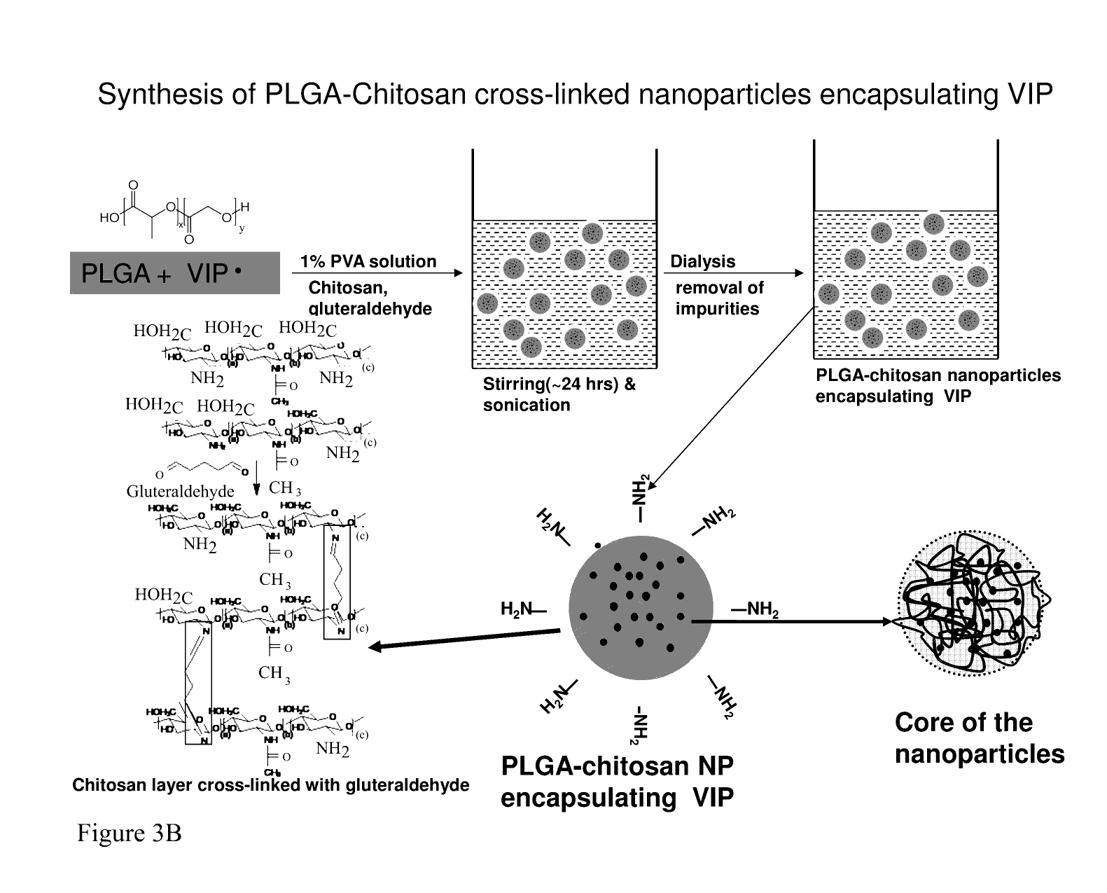

FIGS. 3A and 3B, depict the synthesis and preparation of chitosan-PLGA hybrid nanoparticles with and without VIP. In FIG. 3A, PLGA is mixed with chitosan and PVA(1%) in an overnight stirring and sonication step. Subsequently the mixture undergoes a dialysis step to remove impurities. PVA is used as a stabilizer, while DMSO (0.1% v/v) and acetic acid (0.1% v/v) were incorporated as solvents. These may be removed by the subsequent dialysis step. FIG. 3B shows, PLGA, VIP, chitosan, and gluteraldehyde are mixed together, for approximately twenty-four hours, in a stirring and sonication step. Subsequently the mixture undergoes a dialysis step to remove impurities. The result is a PLGA-chitosan nanoparticle, wherein the chitosan layer is cross-linked with gluteraldehyde.

VIP encapsulated in nanoparticles with different degrees of cross-linking is tested for optimal pharmacokinetics. The formulation is optimized for loading efficiency. The ratios of different constituents are manipulated for optimum delayed release. To achieve that goal, the following parameters are evaluated: Particle size analysis by DLS spectroscopy, zeta potential measurement, in vitro release kinetics, Transmission Electron Microscopy for size confirmation, measurement of VIP inside the nanoparticles (by HPLC or LC/MS).

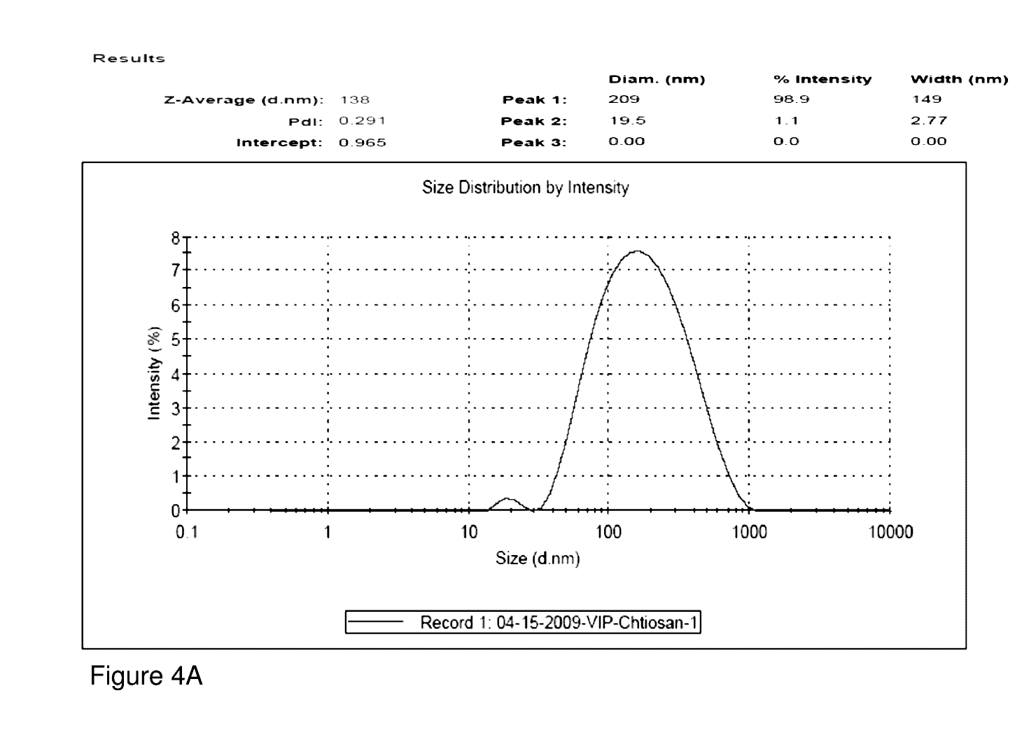

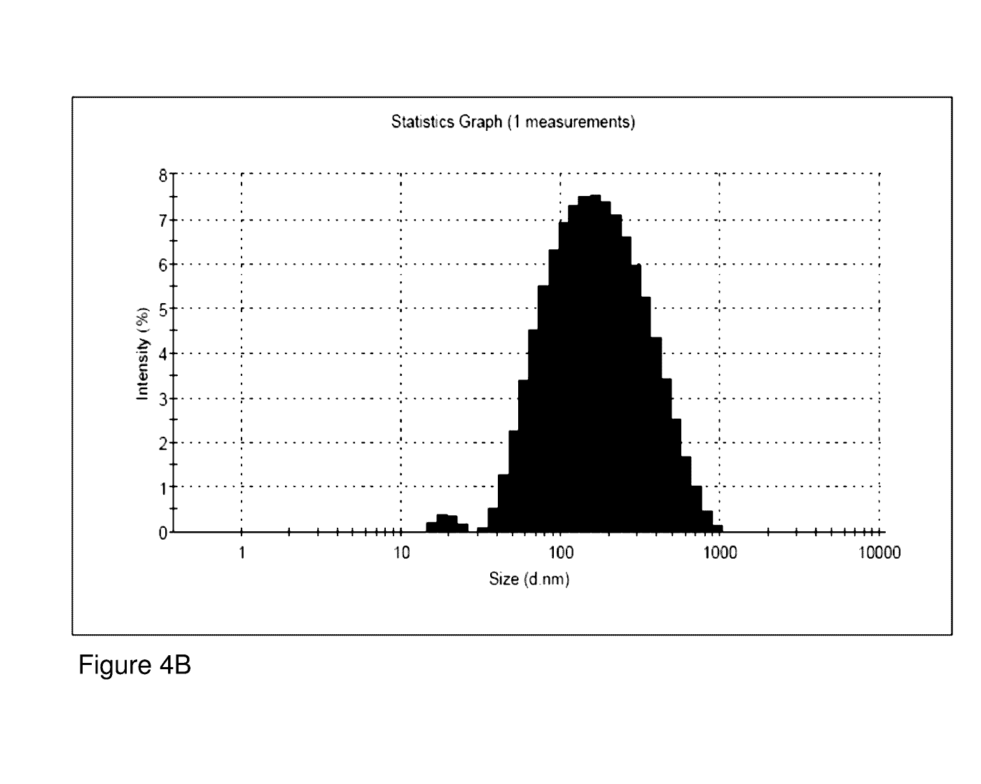

FIGS. 4A and 4B depicts the size measurement of chitosan nanoparticles encapsulating VIP by DLS spectroscopy.

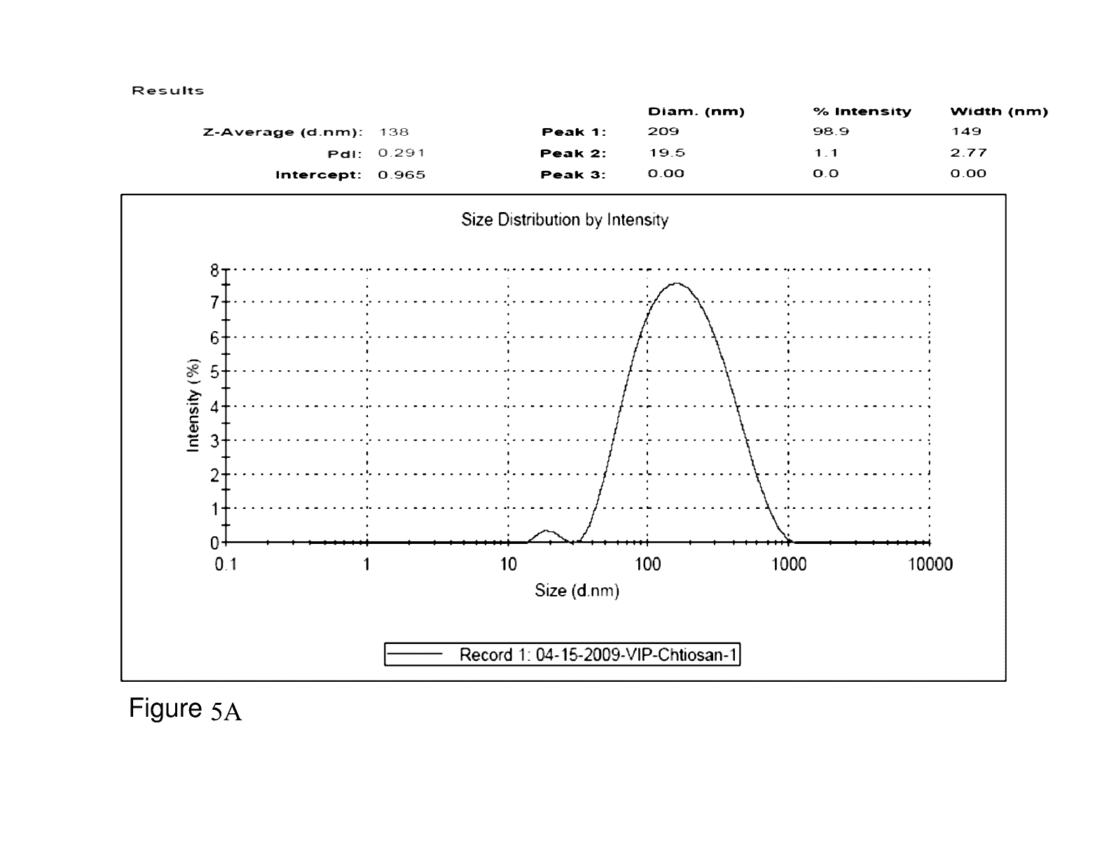

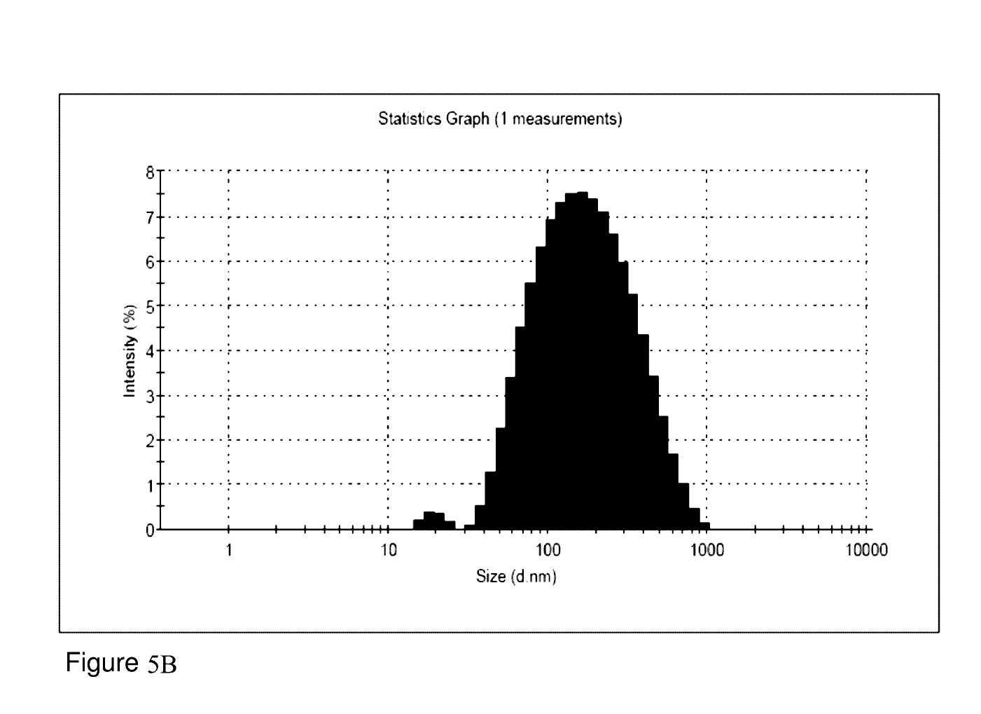

FIGS. 5A and 5B depicts the size measurement of chitosan nanoparticles encapsulating VIP and including gluteraldehyde as a cross-linker.

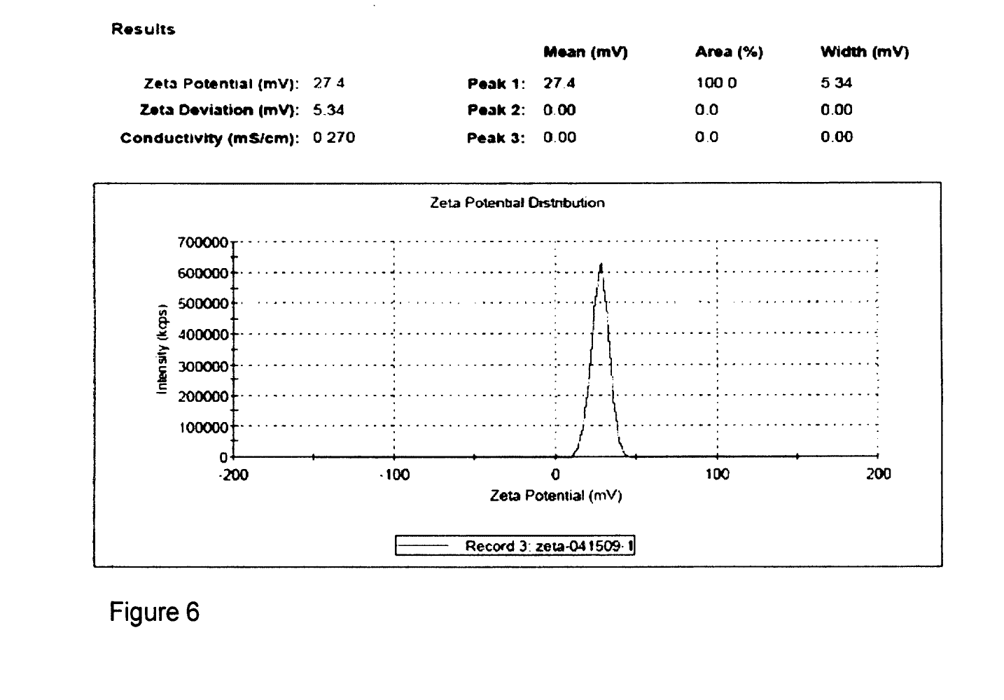

FIG. 6 depicts Zeta potential measurement of chitosan nanoparticles encapsulating VIP.

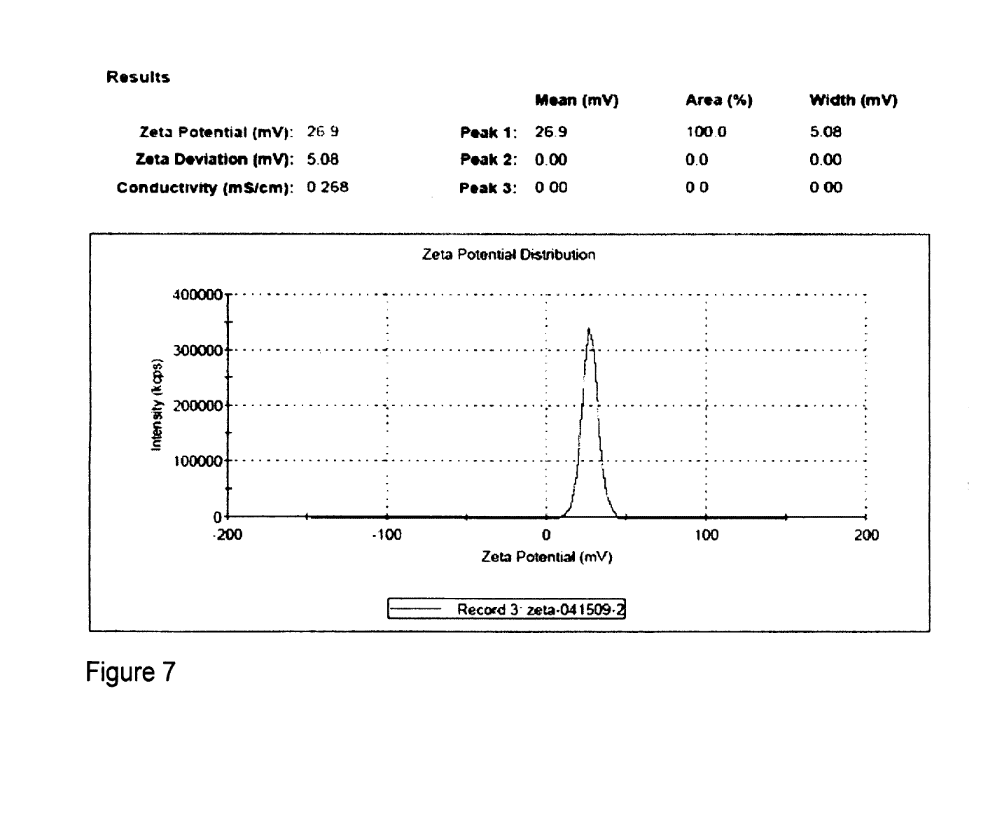

FIG. 7 depicts Zeta potential measurement of void chitosan nanoparticles.

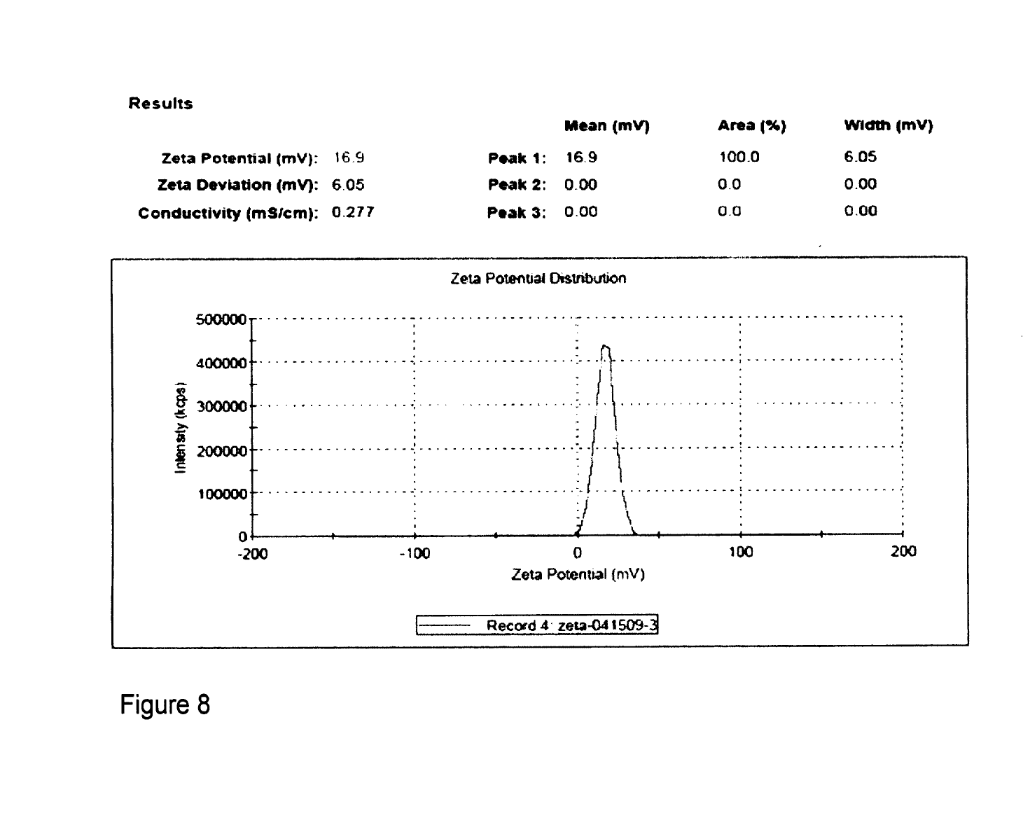

FIG. 8 depicts Zeta potential measurement of gluteraldehyde crosslinked chitosan nanoparticles encapsulating VIP.

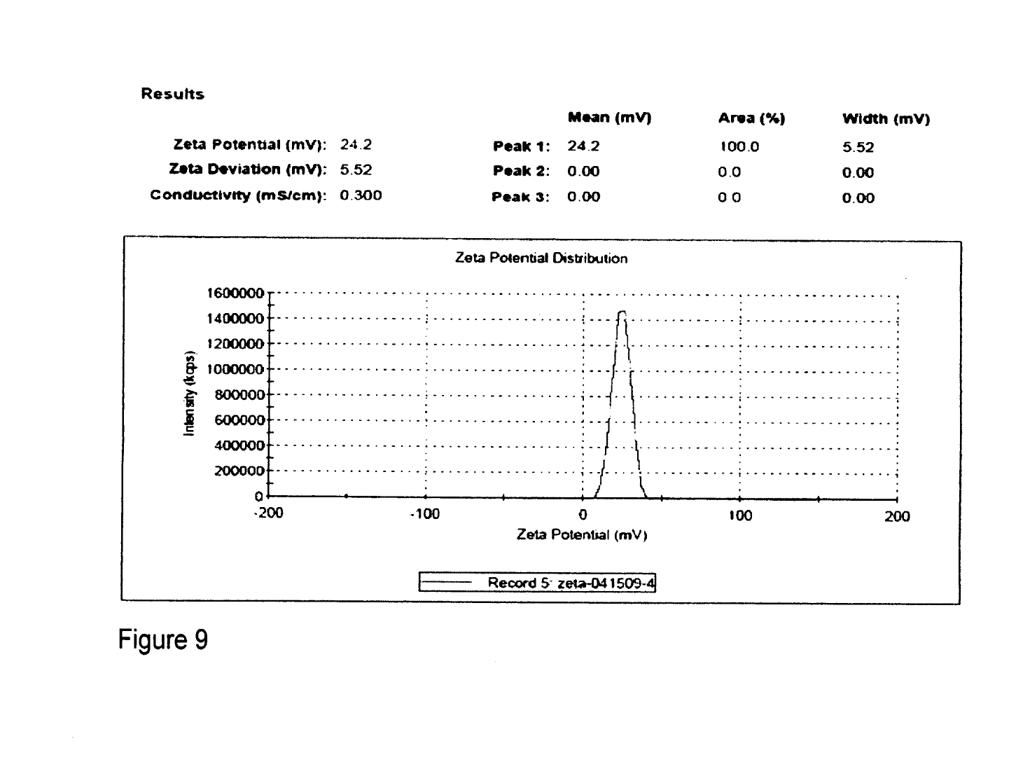

FIG. 9 depicts Zeta potential measurement of gluteraldehyde crosslinked void chitosan nanoparticles.

### Example 3

### Measurement of VIP Loading in Nanoparticles and Delivery to Plasma

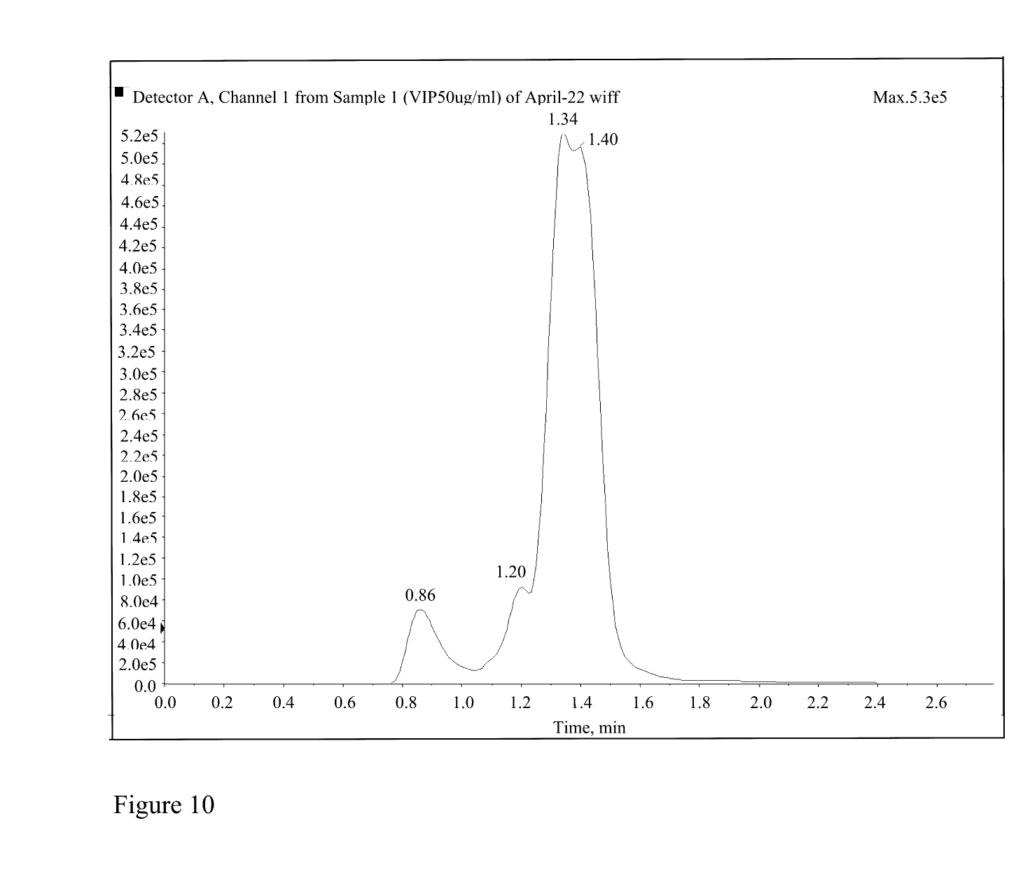

FIG. 10 depicts HPLC measurements of VIP. The values of the peaks are, respectively from lowest to highest: 0.86, 1.20, 1.84, 1.40.

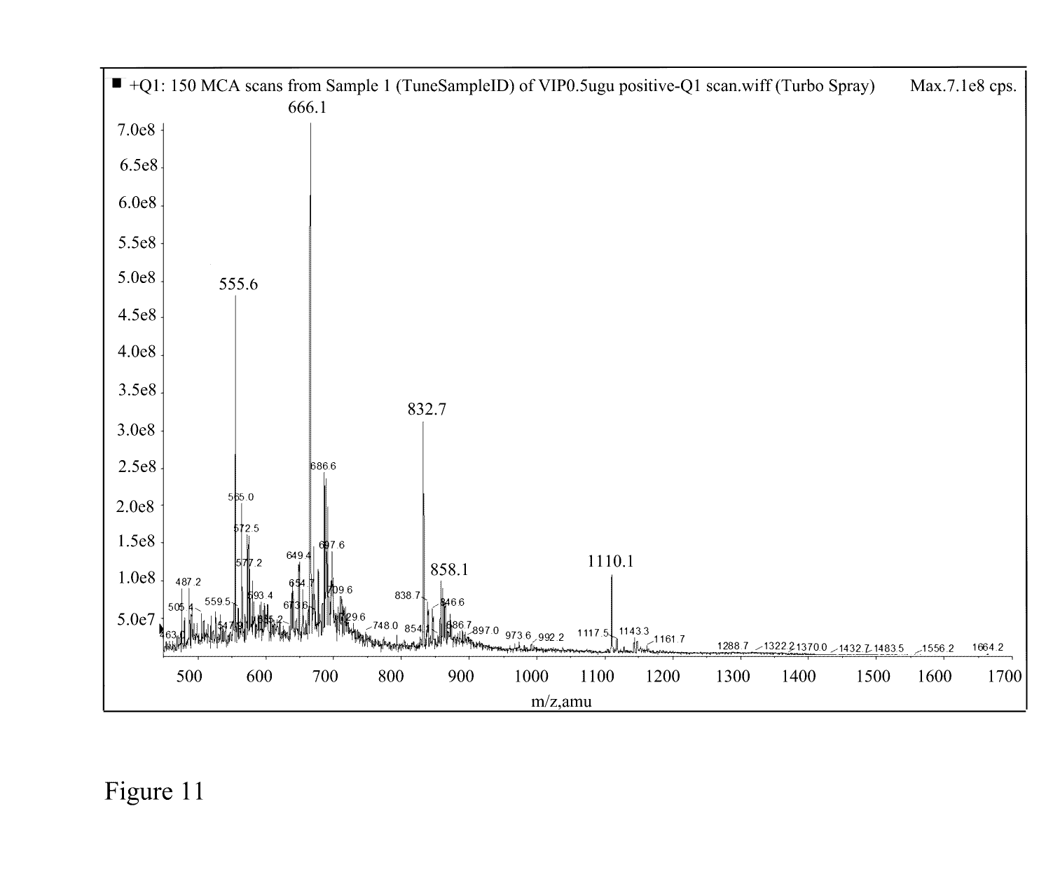

FIG. 11 depicts a Q1 scan of VIP, where the m/z is from 450 to 1700.

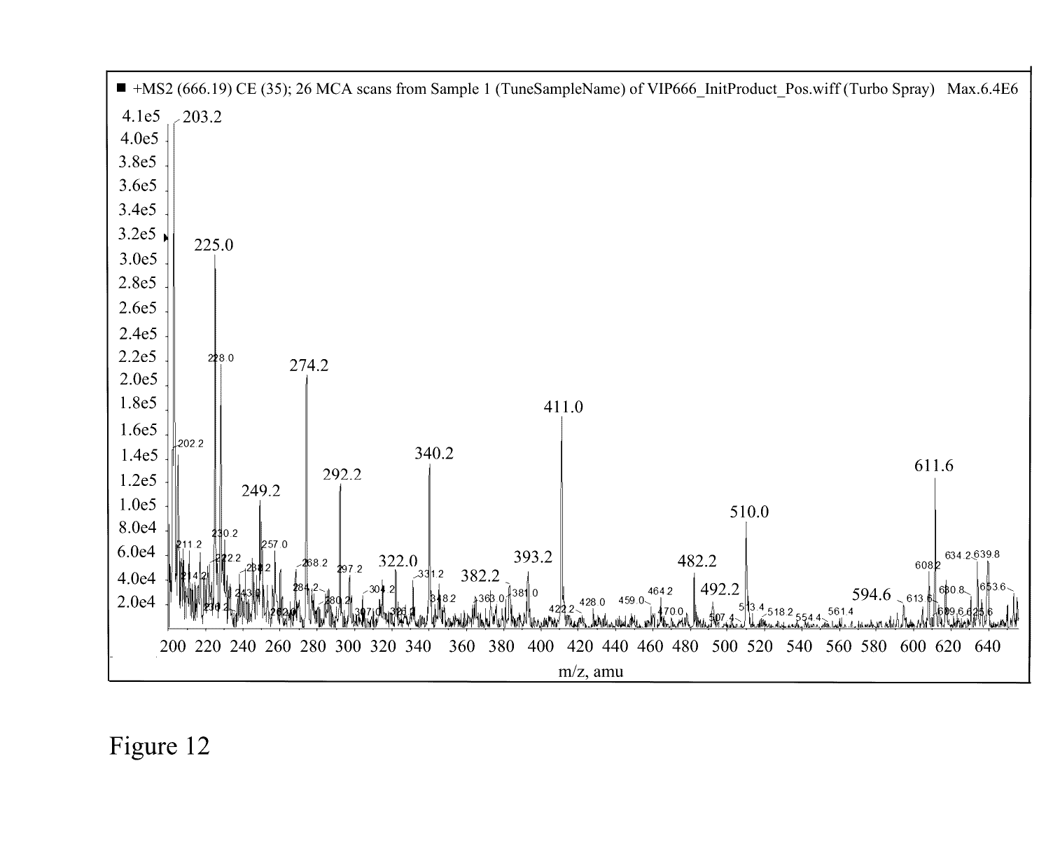

FIG. 12 depicts a Q2 scan of product ion of VIP (M+5H+].

FIG. 12 depicts a VIP standard solution (200 ng/ml in 70% ACN, 0.1% formic acid).

### Example 4

### Synthesis of Hydrophobic Chitosan

Hydrophobic chitosan polymer is synthesized according to the following scheme:

To Chitosan (0.200 g) (75-85% deacetylated) solution in HCl (0.2 N, 20 mL), MeOH (20 mL), NHS, lithocholic acid (106.4 mg, 0.283 mmol) and pyridine (647.0 μL) are added. After overnight stirring at room temperature, another portion of MeOH (40 mL) is added to obtain a clearer reaction mixture. EDAC (81.2 mg, 0.424 mmol) is added and magnetically stirred at room temperature for 24 hrs. Chitosan product is precipitated out by ammonium hydroxide (3 mL) and collected by centrifugation. The precipitates are washed three times with deionized water. The precipitates are then redissolved in 1% AcOH (20 mL), washed with DCM:MeOH (1:4) (3×20 mL), precipitated again with ammonium hydroxide (3 mL), washed with deionized water (3×20 mL) and lyophilized for 48 hours.

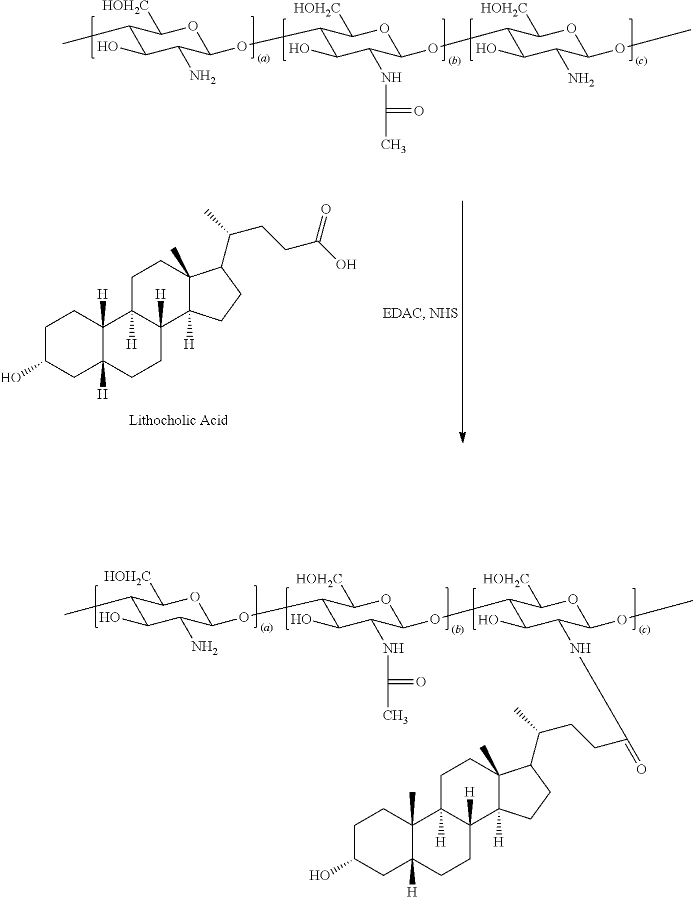

Different ratios of chitosan: lithocholic acid are synthesized.

### Example 5

### VIP in Chorioallantoic Membrane Angiogenesis (CAM) Model

| | #1 | #2 | #3 | #4 | #5 | #6 | #7 | #8 | Mean | Std | SEM |
|---|---|---|---|---|---|---|---|---|---|---|---|
| PBS (Control) | 42 | 30 | 52 | 68 | 58 | | | | 50.0 | 14.6 | 6.5 |
| FGF (2 μg/ml, 20 ng/CAM) | 134 | 112 | 118 | 137 | 130 | 110 | 90 | | 118.7 | 16.5 | 7.4 |
| VIP (0.1 ug/ml, 1 ng/CAM) | 79 | 91 | 88 | 80 | 88 | 81 | 86 | 85 | 84.8 | 4.3 | 1.5 |
| VIP (1 ug/ml, 10 ng/CAM) | 108 | 98 | 97 | 100 | 118 | 93 | 111 | | 103.6 | 9.0 | 3.4 |
| VIP (10 ug/ml, 100 | 129 | 96 | 130 | 137 | 105 | 133 | | | 121.7 | 16.9 | 6.9 |
| ng/CAM) | | | | | | | | | | | |
| Total # of branches [FGF - | 84.0 | 62.0 | 68.0 | 87.0 | 80.0 | 60.0 | 40.0 | | 68.7 | 16.5 | 7.4 |
| PBS(average)] | | | | | | | | | | | |
| Total # of branches [VIP | 29.0 | 41.0 | 38.0 | 30.0 | 38.0 | 31.0 | 36.0 | 35.0 | 34.8 | 4.3 | 1.5 |
| (1 ng/CAM) - | | | | | | | | | | | |
| PBS(average)] | | | | | | | | | | | |
| Total # of branches [VIP | 58.0 | 48.0 | 47.0 | 50.0 | 68.0 | 43.0 | 61.0 | 53.6 | 9.0 | 3.7 | |
| (10 ng/CAM) - | | | | | | | | | | | |
| PBS(average)] | | | | | | | | | | | |
| Total # of branches [VIP | 79.0 | 46.0 | 80.0 | 87.0 | 55.0 | 83.0 | | 71.7 | 16.9 | 6.9 | |
| (100 ng/CAM) - | | | | | | | | | | | |
| PBS(average)] | | | | | | | | | | | |

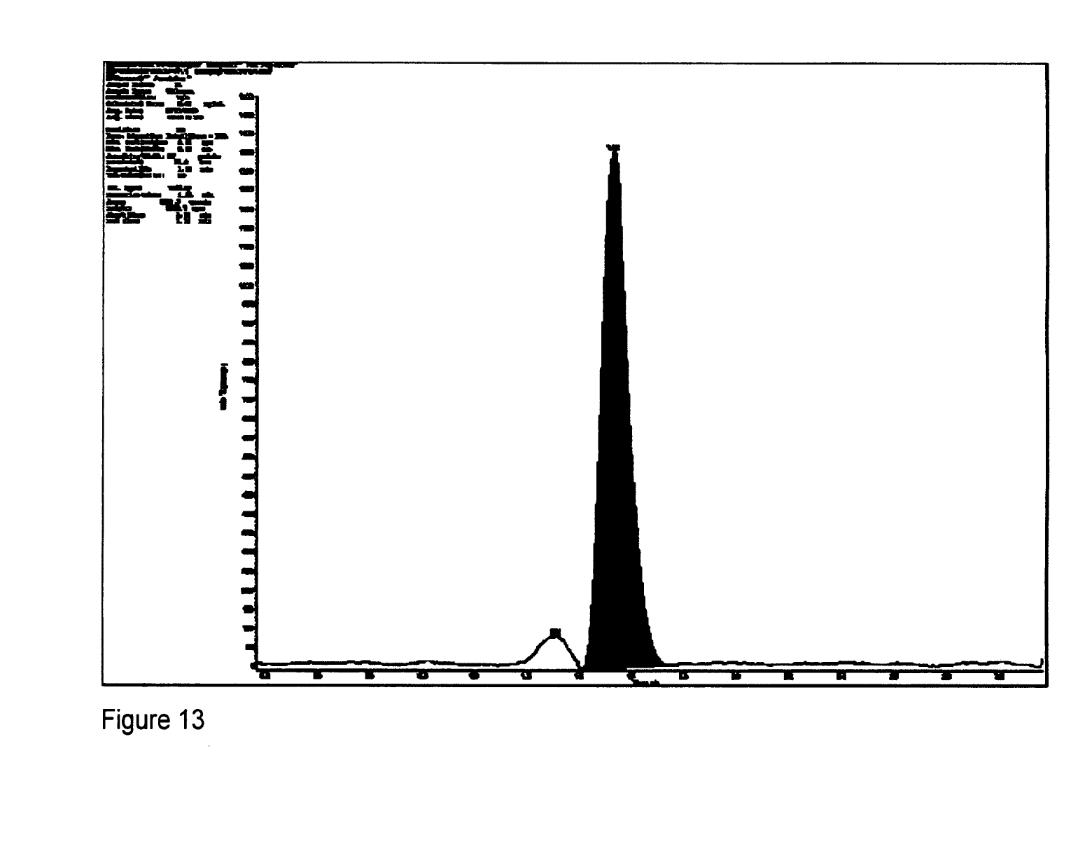

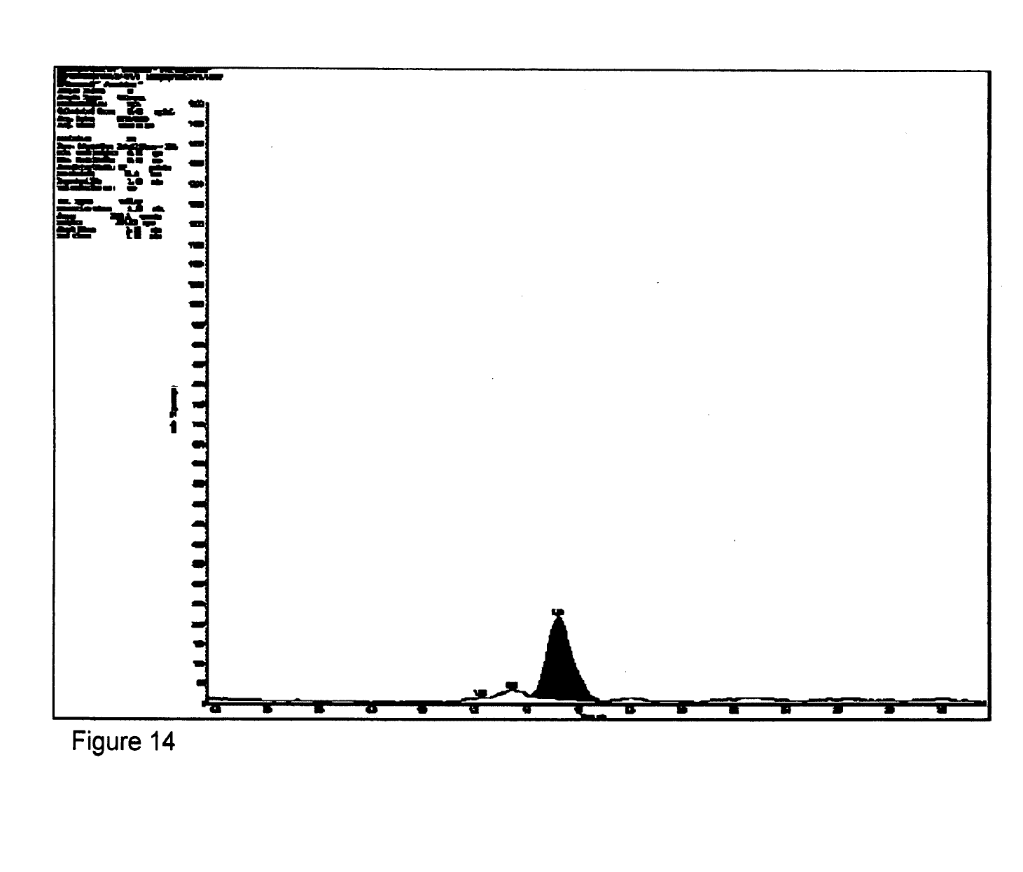

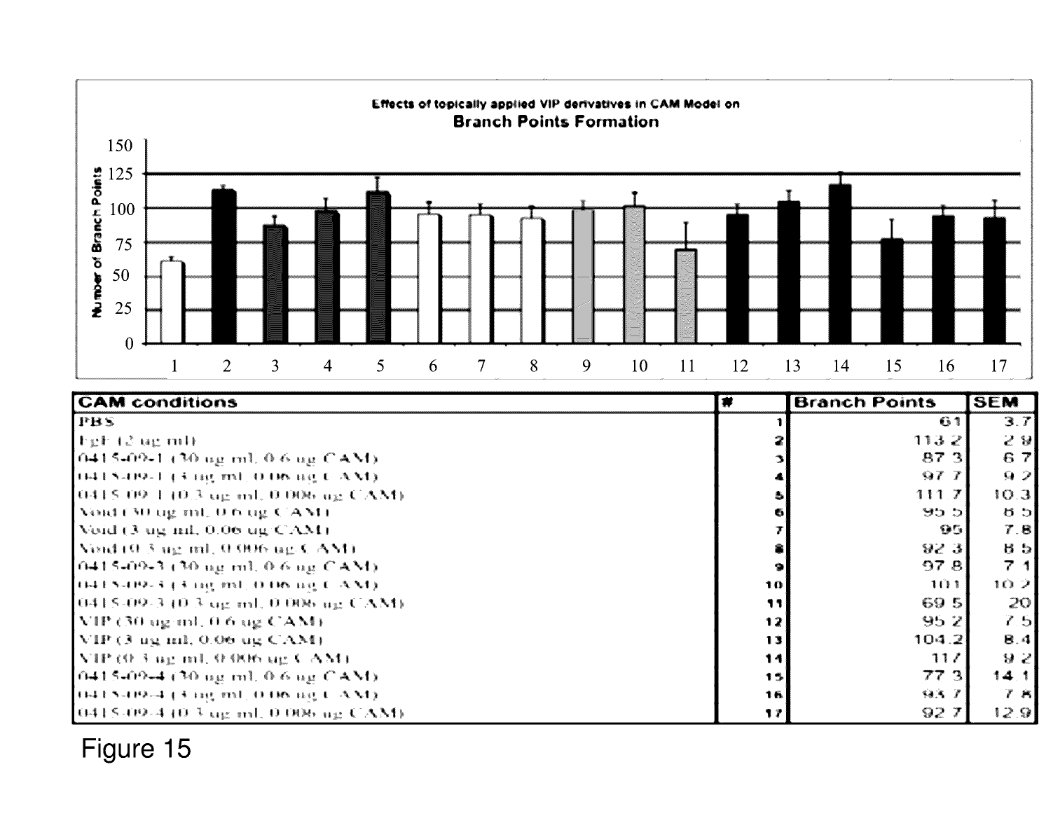

FIG. 13 depicts the results of testing regarding Nanoparticle encapsulates. The testing encompasses the results of branch points formation in the topical application of VIP derivatives in a CAM model.

The examples and drawings provided in the detailed description are merely examples, which should not be used to limit the scope of the claim construction or interpretation.

Alternative combinations and variations of the examples provided will become apparent based on this disclosure. It is not possible to provide specific examples for all of the many possible combinations and variations of the embodiments described, but such combinations and variations may be claims that eventually issue.

1. A formulation comprising VIP nanoparticles, wherein the VIP nanoparticle comprises VIP encapsulated or immobilized on a bioabsorbable polymer.

2. The formulation of claim 1 wherein the polymer comprises chitosan.

3. The formulation of claim 1 wherein the polymer comprises poly(lactic-co-glycolic acid) (PLGA).

4. The formulation of claim 1 wherein the polymer comprises chitosan crosslinked using glutaraldehyde.

5. The formulation of claim 1 wherein the polymer comprises chitosan linked to bile acids.

6. The formulation of claim 1 wherein the polymer comprises chitosan linked to PLGA.

7. The formulation of claim 1 wherein the nanoparticles have an average diameter of 50-1000 nm.

8. The formulation of claim 1 wherein the nanoparticles have a zeta potential of 10-100 mV.

9. The formulation of claim 1 wherein the nanoparticle comprises a second pharmacologically active ingredient.

10. The formulation of claim 1 comprising VIP which is not covalently bound to the polymer.

11. The formulation of claim 1 comprising VIP which is covalently bound to the polymer.

12. The formulation of claim 1 comprising both VIP which is not covalently bound to the polymer and VIP which is covalently bound to the polymer.

13. A composition comprising a VIP nanoparticle formulation according to claim 2 covalently linked to chitosan.

14. The composition of claim 13 wherein the linkage is between the amino groups on the chitosan and the phenolic hydroxy on the VIP.

15.-23. (canceled)

24. A method for treating pulmonary hypertension, comprising administering an effective amount of a VIP nanoparticle formulation according to claim 1 to a patient in need thereof.

25.-33. (canceled)

34. A method for treating cardiac insufficiency, comprising administering a therapeutically effective amount of a VIP nanoparticle formulation according to claim 1 to a patient in need thereof.

35. The method of claim 34, wherein said cardiac insufficiency is heart failure, angina, or acute myocardial infarction.
